(19) United States
(12) Patent Application Publication
(10) Pub. No.: US 2026/0002303 A1
(43) Pub. Date: Jan. 1, 2026

(54) PROCESS FOR MANUFACTURING A POTENTIATING PROTEIN COMPOSITION WITH INCREASED EFFICIENCY AND LONGEVITY AND USES THEREOF

(71) Applicant: 14787285 Canada Inc. dba Nuria Scientific, St. Catharines (CA)

(72) Inventor: Nicholas Leroy Ledra, Thorold (CA)

(21) Appl. No.: 18/756,409

(22) Filed: Jun. 27, 2024

(51) Int. Cl. D04H 1/728; C08K 3/105; C12N 9/02

(52) U.S. Cl. CPC D04H 1/728; C08K 3/105; C12N 9/0061; C12Y 110/03002

(57) ABSTRACT

A nanoparticle composition, including: a nanoparticle; at least one polymer; and at least one protein; wherein: manufacturing the nanoparticle composition includes: combining a nanoparticle solution comprising the nanoparticle and a solvent with the at least one polymer in a first container to create a first solution; and combining the first solution with the at least one protein in a second container; the nanoparticle is selected from a group comprising boron nitride, silica, graphene, cellulose, carbon, latex, silver, and gold; the at least one polymer is selected from a group comprising polyethylene glycol, polyvinyl alcohol, silanes, paraxylene, and phenol formaldehyde; and the at least one protein is selected from a group comprising proteinases, kinases, proteases, laccases, and peroxidases.

### FIELD OF THE DISCLOSURE

This disclosure relates to potentiating protein compositions in general, and more particularly, to increasing the longevity and activity through a composition comprising a nanoparticle, a polymer, and a protein.

### BACKGROUND

Examples of potentiating of proteins use enzymes with antimicrobial compositions to kill or inhibit the growth of microorganisms such as bacteria, fungi, viruses, and parasites. While these substances have beneficial use in various applications, such as medical applications, environmental applications, and household products, room for improvement remains.

Examples of medical applications include personal protective equipment and point-of-care devices. Personal protective equipment in particular may be multi-layered, heavy, bulky, and/or unbreathable. Further, different gowns may be required for different types of threats. As such, a need exists for improved antimicrobial personal protective equipment. For enzymatic point-of-care devices, wherein a blood sample is placed on a lateral flow chip or strip, a protease enzyme used eats itself and other enzymes and subsequently the blood sample along its pathway. Moreover, many enzymes degrade rapidly under ambient conditions, wherein their structure denatures with time rendering the enzymes inoperative. Current solutions are environmentally unfriendly or do not meet the needs of our current reality (i.e., providing antimicrobial solutions). As such, a need exists for improved enzymatic point-of-care devices.

An example of environmental applications includes cleaning of oil spills. Current technology for the cleaning of oil spills involves the use of oil-spill sequestering agents to soak up petroleum product. Oil-spill sequestering agents known in the art use oil-eating microbes, hair, sand, and/or plant fibers to soak up petroleum byproducts. A problem with current oil-spill sequestering agents is petroleum products remain sequestered or trapped within a material, such as paper towel, hair, or coconut fibers which are then disposed as hazardous waste. Further, current technology used in cleaning oil spills only soaks up an oil spill, and there is no means of disposing or reusing the oil once the oil has been sequestered. As such, there is a need to improve current technology used in the cleaning of oil spills.

### SUMMARY

The following presents a simplified summary of some embodiments of the techniques described herein in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented below.

Some aspects provide a nanoparticle composition, including: a nanoparticle; at least one polymer; and at least one protein; wherein: manufacturing the nanoparticle composition includes: combining a nanoparticle solution comprising the nanoparticle and a solvent with the at least one polymer in a first container to create a first solution; and combining the first solution with the at least one protein in a second container; the nanoparticle is selected from a group comprising boron nitride, silica, graphene, cellulose, carbon, latex, silver, and gold; the at least one polymer is selected from a group comprising polyethylene glycol, polyvinyl alcohol, silanes, paraxylene, and phenol formaldehyde; and the at least one protein is selected from a group comprising proteinases, kinases, proteases, laccases, and peroxidases.

Some aspects include a process for manufacturing a nanoparticle composition, including: combining a nanoparticle solution including a nanoparticle and a solvent with at least one polymer in a first container to create a first solution; and combining the first solution with at least one protein in a second container; wherein: the nanoparticle composition includes: the nanoparticle; the at least one polymer; and the at least one protein; the nanoparticle is selected from a group comprising boron nitride, silica, graphene, cellulose, carbon, latex, silver, and gold; the at least one polymer is selected from a group comprising polyethylene glycol, polyvinyl alcohol, silanes, paraxylene, and phenol formaldehyde; and the at least one protein is selected from a group comprising proteinases, kinases, proteases, laccases, and peroxidases.

### BRIEF DESCRIPTION OF THE FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates an example of a process for forming an electrospun textile, according to some embodiments.

FIGS. 2A and 2B illustrate an example of a process for forming an electrospun textile using a combination of two or more distinct solutions with distinct properties, according to some embodiments.

FIG. 3 illustrates an example of a nanoparticle composition, according to some embodiments.

FIG. 4A illustrates a process for manufacturing a conjugated nanoparticle composition, according to some embodiments.

FIG. 4B illustrates a process for coating a sponge with the conjugated nanoparticle composition described in FIG. 4A, according to some embodiments.

FIG. 5 illustrates Fourier-Transform Infrared Spectroscopy (FTIR) analysis of oil and the coated sponge described in FIG. 4B submerged in oil.

FIG. 6 illustrates visible ultraviolet (UV-vis) spectroscopy analysis of absorption of oils using the coated sponge described in FIG. 4B.

### DETAILED DESCRIPTION OF SOME EMBODIMENTS

The present inventions will now be described in detail with reference to a few embodiments thereof as illustrated in the accompanying drawings. In the following description, numerous specific details are set forth in order to provide a thorough understanding of the present inventions. It will be apparent, however, to one skilled in the art, that the present invention may be practiced without some or all of these specific details. In other instances, well known process steps and/or structures have not been described in detail in order to not unnecessarily obscure the present invention. Further, it should be emphasized that several inventive techniques are described, and embodiments are not limited to systems implanting all of those techniques, as various cost and engineering trade-offs may warrant systems that only afford a subset of the benefits described herein or that will be apparent to one of ordinary skill in the art.

Embodiments provide a nanoparticle composition comprising at least a nanoparticle, at least one protein, and at least one polymer. In some embodiments, the nanoparticle composition is a conjugated nanoparticle composition wherein the at least one protein (e.g., an enzyme or collection of enzymes) and/or the at least one polymer is conjugated to the nanoparticle. The main purpose of conjugating the at least one protein, such as an enzyme or collection of enzymes, is to stabilize the enzyme or collection of enzymes, outside of an organism from which it was isolated, for use in industrial, commercial, or other applicable applications. Stabilized proteins or enzymes have increased stability for harsh conditions used in industrial processes, medicinal uses, food processing, filtration and separation processes, energy sequestration and storage, cosmetics and skin care, etc.

In some embodiments, the nanoparticle composition is a liquid. In some embodiments, the nanoparticle composition is a solid. Examples of solids include a textile, a sponge, a foam, a hydrogel, a gel, a colloid, a hydrofilm, a suspension, and a biogel. In some embodiments, the nanoparticle composition is a solid comprising a textile, wherein the textile comprises the nanoparticle, the at least one protein, and the at least one polymer. In some embodiments, the textile is electrospun from a colloidal suspension comprising the nanoparticle composition. In some embodiments, the electrospun textile exhibits antimicrobial, anti-chemical, and radiation attenuating properties. In some embodiments, the electrospun textile limits microbial growth on a surface of the textile, limits chemicals from penetrating through the textile and onto skin of a wearer, and attenuates radiation levels between 0.5 to 15 KeV. In some embodiments, the textile exhibits at least one of the following characteristics: deflection and/or attenuating properties for certain types of radiation (e.g., electromagnetic, microwave, infrared, UV radiation, certain degrees of alpha and beta waves, etc.); filtration pores (e.g., 0.05 nm-0.1 mm in size) to be able to selectively filter certain chemicals; and inhibition of growth of micro-organisms (e.g., bacteria, virus, fungi, etc.). The textile may be comprised of a single layer or multiple layers of a same textile or different combination of textiles layered one on top of the other. A thickness of each textile layer may vary in different embodiments.

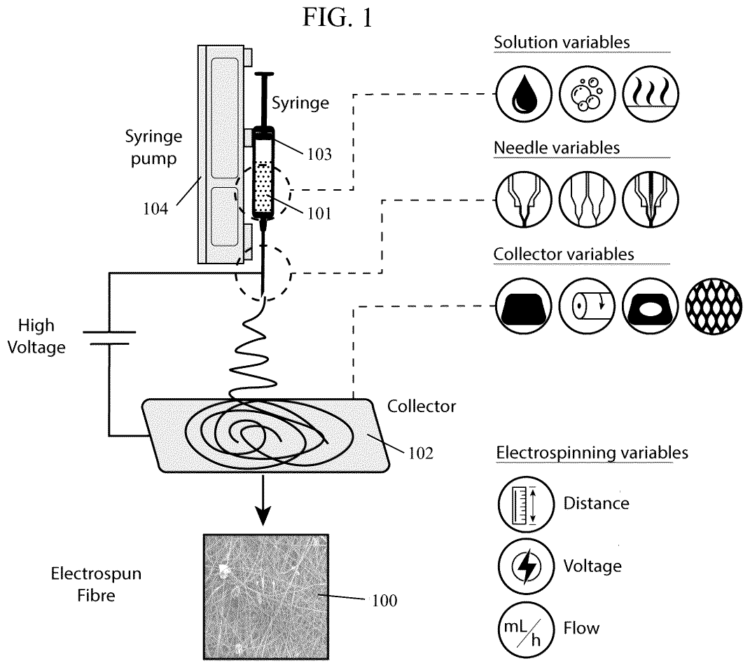

FIG. 1 illustrates an example of an electrospinning process for manufacturing an electrospun textile 100 including electrospinning a solution of a nanoparticle composition 101 at a controlled rate of deposition onto a collector 102 and collecting the electrospun fiber 100. The process may require loading a syringe 103 with different solutions of nanoparticle compositions, each having a different polymer solution concentration (e.g., 4, 10, 15 wt. %), and choosing the solution of nanoparticle composition with the particular polymer concentration that best flows. In one embodiment, the solution is a viscous liquid, but not a gel. The process may further require loading the syringe 103 with the solution of nanoparticle composition 101 and setting a pumping speed of a syringe pump 104 such that any bead of the solution of the nanoparticle composition 101 wiped from a tip of the syringe 103 is immediately replaced. The process may further require grounding the collector 102 and attaching a high voltage wire from the tip of the syringe 103 to a conductor plate (e.g., a small square of conductive material such as aluminum foil through which the syringe tip protrudes). The process may further require starting electrospinning and observing a stream 105 of the solution of nanoparticle composition 101 coming out of the syringe 103 for signs of fiber or filament extrusion and formation, wherein a voltage is slowly ramped up and the bead of the solution of nanoparticle composition 101 at the tip of the syringe 103 is observed. The process may further require adjusting the voltage to obtain a long and steady stream of the solution of nanoparticle composition 101, and if a steady stream cannot be obtained, adjusting the polymer solution concentration. Examples of polymer solution concentrations include, for example, 10% poly-L-lactic acid (PLA) in tetrahydrofuran (THF), 10% polycaprolactone in THF or 10% Polyacrylonitrile in dimethyl formamide. Examples of the collector 102 include, for example, aluminum foil or plastic film as the deposition substrate, where a formed textile may be easily detached. In some embodiments, a formed textile thickness includes 25 g/m² non-woven fabric having a thickness between 100 to 200 mm (e.g., 175 mm). Some embodiments include random fiber arrangement on the collector, optimally on a rotating drum collector. In some embodiments, two or more syringes are used. For instance, where two syringes are used, a first syringe pump may be set to a diameter of 14.96 mm and a rate of 1500 ul/h and the second syringe pump may be set to a diameter of 12.45 mm and a rate of 500 ul/h. Typical voltage settings between a collector and two syringes include, for a first syringe: HV 1, SP −1.0 kV, PV 0.0 kV, and for a second syringe: HV 2, SP+14.0 kV, PV 0.0 kV. In some embodiments, a distance between the tip of the syringe from a top surface of the collector is 200 mm. In embodiments where a rotating collector is used, the rotational speed of the rotating collector may be 50 mm/s.

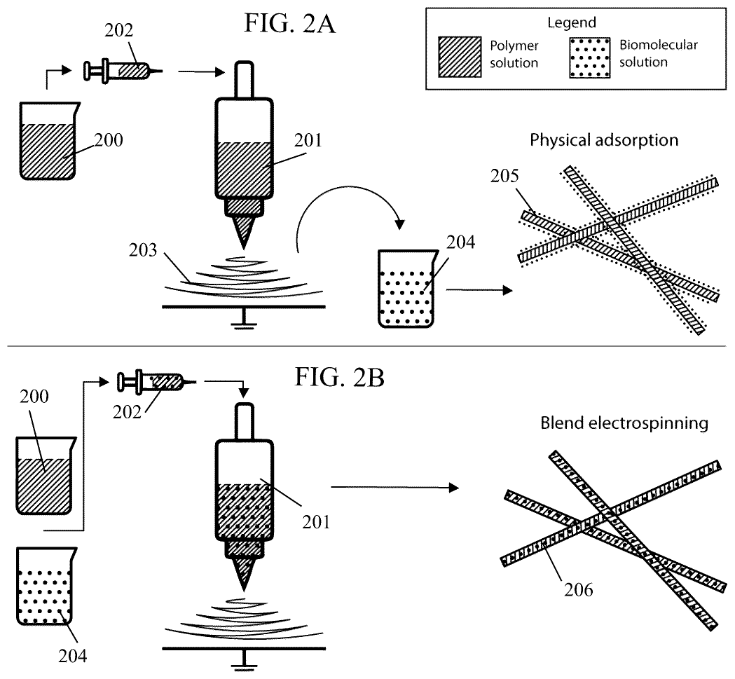

FIGS. 2A and 2B illustrate an apparatus and method for creating functionalized nanofibers. In FIG. 2A, a polymeric solution 200 is introduced to an electrospinning injector 201 using a syringe 202. The electrospinning injector 201 produces fibers 203 from electrospinning the polymeric solution 200. The fibers 203 are added to a mixture 204 (e.g., nanoparticle, solvent, amino acids, essential oils, lipid, sugar, and/or protein) that can be in the form of a solution, a colloid, a suspension, a hydrogel, or a gel-sol. The resulting mixture is physically adsorbed with time/centrifugation and then filtered to separate the conjugated fibers 205. In FIG. 2B, the polymeric solution 200 and the mixture 204 are mixed in one vessel, then the resulting mixture is introduced to the electrospinning injector 201 using the syringe 202. The resulting mixture is electrospun to produce conjugated fibers 206.

In embodiments, the nanoparticle comprises an inert nanosized particle, such as silica, graphene, cellulose, carbon, latex, etc. In some embodiments, the nanoparticle is selected from a metal, such as silver and gold. In some embodiments, the nanoparticle comprises silica or is coated with silica. In some embodiments, the silica surface of the nanoparticle is modified with 3-aminopropyl-tri-ethoxysilane to produce free amine bond linkages or another type of silane or silicate, such as tetraethyl orthosilicate. In some embodiments, the at least one protein is covalently bonded to the nanoparticle through a sulfuryl and amide linkage group. In some embodiments, the nanoparticle exhibits at least one of the following characteristics: a volume of 50 micron³ (similar to the size of a red blood cell); an irregular pore size ranging from nanometers to microns; a surface area between 50 to 600 m²/g; a density between 160 to 190 kg/m³; and a pH of 7. In some embodiments, the nanoparticle is stored between −5 to −20 degrees Celsius. In some embodiments, surface characteristics of the nanoparticle includes at least one of: pores of differing sizes (e.g., 0.1 to 1 nm) and a porous outer surface. In some embodiments, a layered structure of the nanoparticle encapsulates a fluorophore (i.e., a photo synthetic dye that can be conjugated to a protein or another molecule), a protein, or a nanoparticle (e.g., silica and a metal such as silver and gold).

In some embodiments, the at least one protein comprises an enzyme, preferably selected from the group including proteinases, kinases, proteases, laccases, and peroxidases. The enzyme may also be selected from other groups, such as amylase, cellulase, hemi-cellulase, DNAse, RNAse, pectinase, proteinase, and lipase.

In some embodiments, the at least one polymer is selected from the group including polylactic acid, polyethylene glycol, and combinations thereof. The polymer may also be selected from other groups, such as linker polymers including polyvinyl alcohol and polyethylene glycol.

Some embodiments provide an antibacterial nanoparticle composition including non-porous silica, a bi-functional covalent polyethylene glycol linker, and at least one enzyme. The antibacterial nanoparticle composition was tested for antibacterial activity. Three sets of specimens, each specimen having a size of 50 mm×50 mm and a thickness between 1 to 50 mm, were tested for antibacterial activity. One set of specimens was a control (i.e., untreated with an antibacterial nanoparticle composition) while the remaining two sets of specimens were treated with the antibacterial nanoparticle composition. The treated sets of specimens were covered with a film of 40×40×0.05 mm plastic. All three sets of specimens were then exposed to Staphylococcus aureus ATCC 6538P (S. aureus) or Escherichia coli ATCC 8739 (E. coli) and incubated for 24 hours at 35° C. and 90% relative humidity. The starting inoculum concentration of S. aureus was 5.8 E5 and E. coli was 6.3 E5. The amount of inoculum used was 0.4 ml per sample, wherein each sample was exposed to one type of bacterial strain. Results following ISO 22196 standard test method (other than the deviation of testing in one replicate only) show for the control set of specimens at 24h a mean of viable S. aureus level was 2.64 units of bacterial growth while for the treated set of specimens was 0.20 units of bacterial growth. The results indicated an antibacterial activity value R of 3.84 with a % reduction of 99.98. Results following ISO 22196 standard test method (other than the deviation of testing in one replicate only) show for the control set of specimens at 24h a mean of viable E. coli level was 5.71 units of bacterial growth while for the treated set of specimens was −0.20 units of bacterial growth. The results indicated an antibacterial activity value R of 5.91 with a % reduction of 99.9998.

Some embodiments provide a point-of-care device comprising at least the nanoparticle composition described herein. Some embodiments provide personal protective equipment comprising at least the nanoparticle composition described herein. Some embodiments provide an antimicrobial disinfectant comprising at least the nanoparticle composition described herein. Some embodiments provide an antimicrobial disinfectant coating comprising at least the nanoparticle composition described herein.

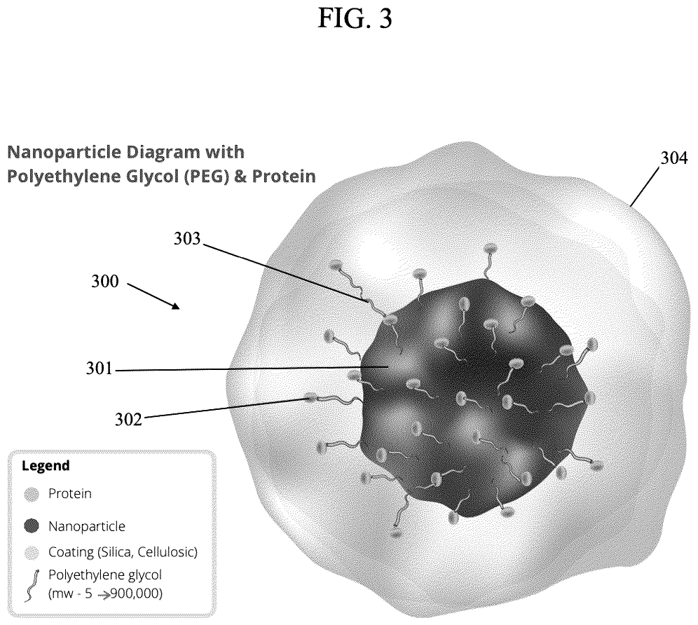

FIG. 3 illustrates an example of an antimicrobial disinfectant comprising a nanoparticle composition 300 including a nanoparticle 301 crosslinked with a protein 302 by polyethylene glycol (a crosslinking polymer) 303. The nanoparticle composition has a coating 304.

In some embodiments, the at least one protein in the conjugated nanoparticle composition is a stabilized protein, such as a stabilized enzyme, wherein the at least one protein is conjugated to the nanoparticle in the conjugated nanoparticle composition to stabilize the at least one protein. The stabilized enzyme may have an extended active life span and exhibit greater activity in comparison to to a non-stabilized enzyme. Increased stability of the enzyme is beneficial under harsh conditions used in, for example, industrial uses, medicinal uses, food processing, filtering processes, and other commercial uses for enzymes and oil/gas extraction and waste water (i.e., pollution) clean-up. In some embodiments, the conjugated nanoparticle composition comprises boron nitride (i.e., the nanoparticle), a crosslinking agent such as polyethylene glycol (i.e., the at least one polymer), and a protein such as laccase (i.e., the at least one protein), wherein conjugation of boron nitride to the protein stabilizes the protein. The boron nitride may be hexagonal boron nitride (hBN), preferably hBN powder. The crosslinking agent may be a crosslinking polymer, such as polyethylene glycol, polyvinyl alcohol, silanes, paraxylene, phenol formaldehyde, or even other enzymes, polysaccharides, or DNA/RNA. The protein may be an enzyme, such as an enzyme selected from the group including proteinases, kinases, proteases, laccases, and peroxidases. The enzyme may also be selected from other groups, such as amylase, cellulase, hemi-cellulase, DNAse, RNAse, pectinase, proteinase, and lipase.

Some embodiments provide an oil sequestering and filtering sponge comprising the conjugated nanoparticle composition described above. In some embodiments, the oil sequestering and filtering sponge is coated with the conjugated nanoparticle composition described above.

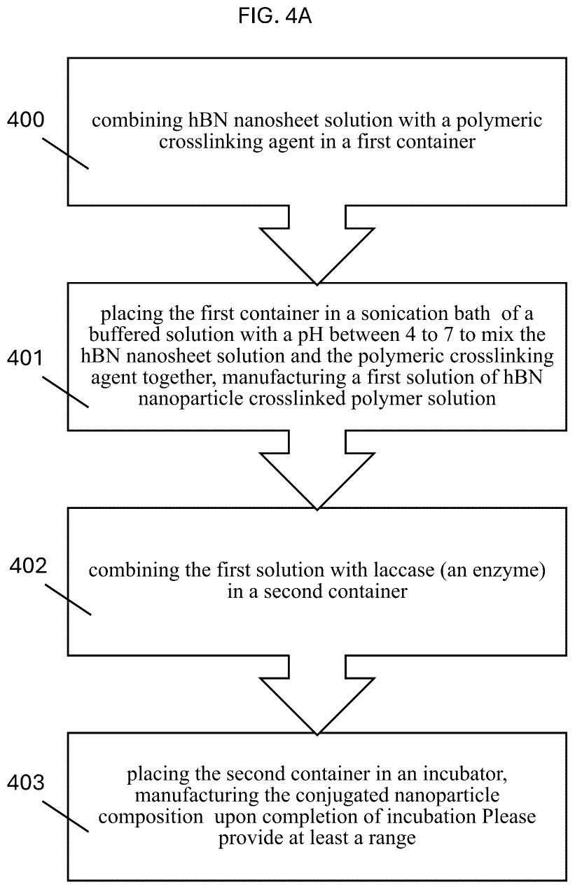

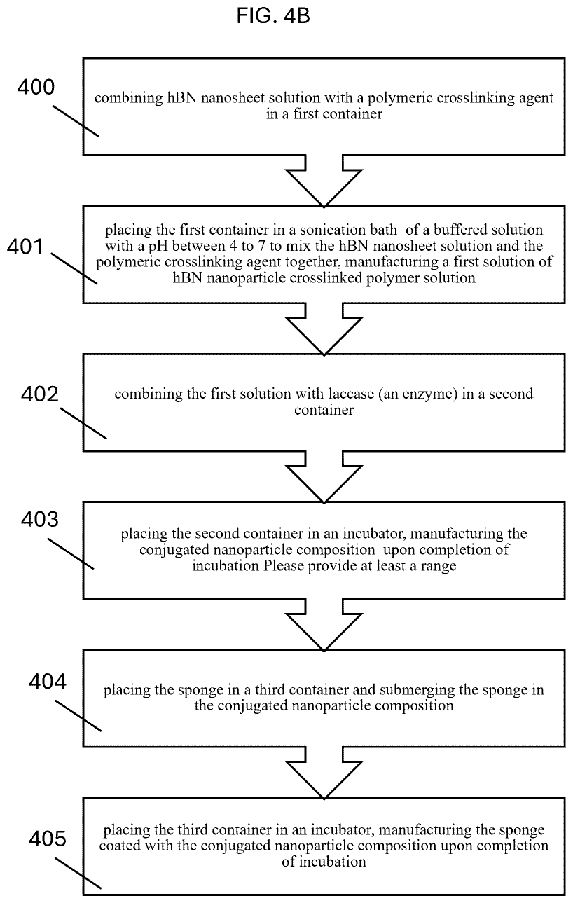

FIG. 4A illustrates a process for manufacturing a conjugated nanoparticle composition, including: (400) combining hBN nanosheet (i.e., a nanoparticle) solution with a polymeric crosslinking agent (i.e., a polymer such as polyethylene glycol, polyvinyl alcohol, etc.) in a first container; (401) placing the first container in a sonication bath of a buffered solution (e.g., phosphate) with a pH between 4 to 7 to mix the hBN nanosheet solution and the polymeric crosslinking agent together, manufacturing a first solution of hBN nanoparticle crosslinked polymer solution; (402) combining the first solution with laccase (an enzyme) in a second container; (403) placing the second container in an incubator, manufacturing the conjugated nanoparticle composition (i.e., a bio-functionalized hBN nanoparticle enzyme solution) upon completion of incubation. The first container may be placed in the sonication bath for between 30 minutes to 2 hours at a temperature between 15 to 50 degrees Celsius. The second container may be placed in the incubator for between 30 minutes to 2 hours at a temperature between 20 to 60 degrees Celsius and a pressure between 46 to 60 kPa. Steps of the process for manufacturing the conjugated nanoparticle composition may be omitted, altered (e.g., altering the crosslinking agent, protein, etc. used), and reordered. Steps may also be added to the process for manufacturing the conjugated nanoparticle composition.

FIG. 4B illustrates a process for coating a sponge (e.g., a melamine sponge) with a conjugated nanoparticle composition, including: (404) manufacturing the conjugated nanoparticle composition, as per the process outlined in (400)-(403) in FIG. 4A; (405) placing the sponge in a third container and submerging the sponge in the conjugated nanoparticle composition; and (406) placing the third container in an incubator, manufacturing the sponge coated with the conjugated nanoparticle composition upon completion of incubation. The submerged coated sponge may be incubated between 5 to 48 hours at a temperature between 20 to 40 degrees Celsius and at a pH between 4 to 7. Steps of the process for coating the sponge with the conjugated nanoparticle composition may be omitted, altered (e.g., altering the crosslinking agent, protein, etc. used), and reordered. Steps may also be added to the process for coating the sponge with the conjugated nanoparticle composition.

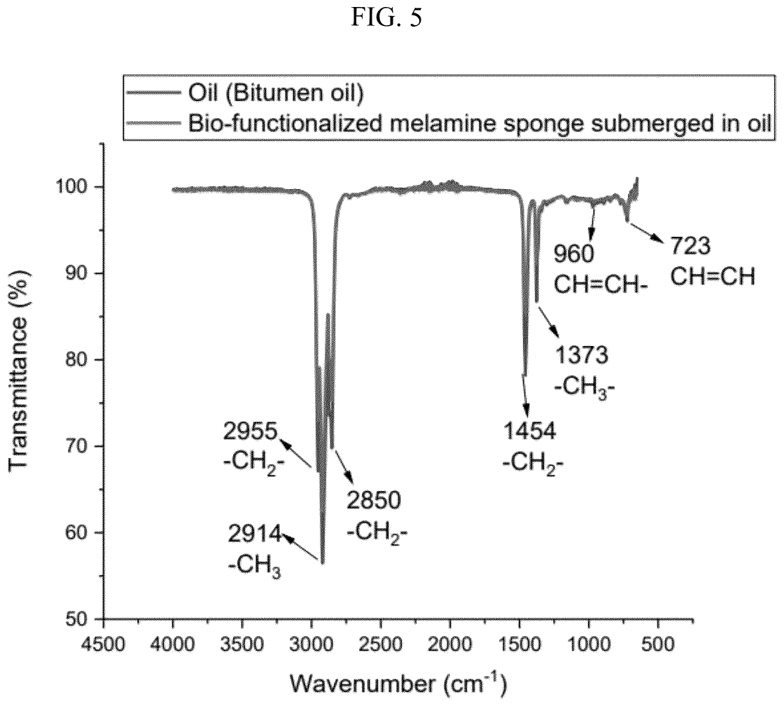

FIG. 5 illustrates FTIR analysis of oil and the coated sponge described in FIG. 4B submerged in oil. The FTIR analysis confirms the coated sponge described in FIG. 4B absorbed the oil. Overlap of the lines of the oil and the coated sponge indicates the coated sponge soaked up the oil. The presence of functional groups was also confirmed via FTIR analysis.

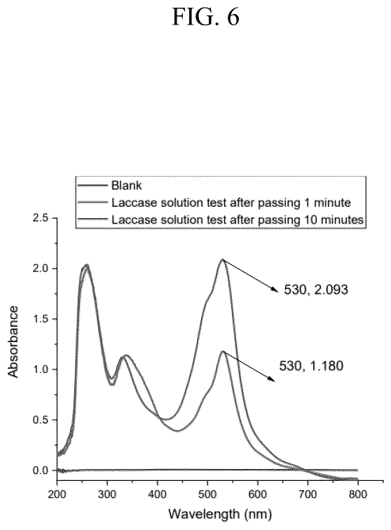

FIG. 6 illustrates visible ultraviolet (UV-vis) spectroscopy analysis of absorption of oils using the coated sponge described in FIG. 4B. The graph exhibits enzyme activity, wherein petroleum products are broken down via the active laccase enzyme even after functionalization onto hBN nanosheets. The graph demonstrates spectrophotometrically the amount of petroleum that has been broken down into smaller chain fatty acids as time progressed from 1 to 10 minutes.

In some embodiments, the conjugate nanoparticle composition comprises silica dioxide (the nanoparticle), polyethylene glycol (the at least one polymer), and protease (the at least one protein). Molecular weights of components of the nanoparticle conjugate composition may include 60.084 g/mol for silica dioxide, 20000 g/mol for polyethylene glycol (may vary between 5-900000 g/mol molecular weight), and 10000 g/mol of protease to 400,000 g/mol of polyethylene glycol. A nanoparticle other than silica dioxide may be used, such as metal, graphene, latex, or cellulose. A protein other than protease may be used, such as any small polypeptide (e.g., 20 amino acid-base pairs).

Some embodiments include a process for manufacturing a conjugate nanoparticle composition, including: (a) forming a nanoparticle mixture and irradiating the nanoparticle mixture (e.g., UV irradiating, preferably UV-A irradiating), wherein the nanoparticle mixture includes: a metal salt (e.g., silver nitrate); an activated radical initiator (e.g., a UVA activated radical initiator, preferably I-2957); and a nanoparticle stabilizer (e.g., cyclohexylamine). In some embodiments, the stabilizer is in the presence of a solvent, such as a polar aprotic solvent selected from the group including acetonitrile, dimethylformamide, and tetrahydrofuran. The solvent may be hydrophobic or hydrophilic. In some embodiments, the nanoparticle includes at least one nanoparticle, preferably a plurality of nanoparticles, of diameter from approximately 1 to 5 nms. In some embodiments, the at least one nanoparticle is spherical in shape. In one embodiments, the nanoparticle mixture is UVA irradiated at 365 nm at 100 W/m² over a period of 10 minutes, preferably under inert atmosphere conditions.

Some embodiments include a process for manufacturing a polymer based conjugate nanoparticle composition, including (a) above, and further including: (b) adding a polymer to the irradiated nanoparticle mixture; and (c) dissolving the polymer in the solvent. The polymer may be selected from the group including polymethylmethacrylate and polycaprolactone. In some embodiments, the polymer is a blend of polymethylmethacrylate and polycaprolactone with a polymethylmethacrylate:polycaprolactone molar ratio between 0:1 to 1:0. In some embodiments, the polymer is dissolved in the solvent by sonication or ultrasonication at a temperature between 30° C. to 35° C. and ambient atmosphere (i.e., air atmosphere), resulting in the polymer based nanoparticle composition. In one embodiment, the metal salt is in the amount of one chemical equivalent, the activated radical initiator is in the amount of one chemical equivalent, the nanoparticle stabilizer is in the amount of 10 chemical equivalents, the solvent is in the amount of 1 to 50 ml, and the polymer is in the amount of approximately 1 to 15 percent by weight. In one embodiment, the polymer based nanoparticle composition has a nanoparticle concentration of 0.001 to 0.01 moles per liter. In another embodiment, the nanoparticle concentration includes any trace amounts of unreacted metal salt. Some embodiments provide an electrospun textile with antimicrobial properties comprising the polymer based nanoparticle composition described herein.

Some embodiments include a process for manufacturing a nanoparticle composition, in particular a cross linking polymer and enzyme of the nanoparticle composition, wherein the cross linking polymer is bifunctional with one end covalently bonded onto a nanoparticle and the other end bonded to a protein, such as an enzyme. In some embodiments, the process includes: determining an isolated/purified enzyme amino acid sequence; determining locations of possible attachment sites for a cross linking polymer (e.g., via OH, COOH, SH, SO, CO, H, NHₓ, or other sites); selecting the cross linking polymer; equilibrating reagents (i.e., enzyme and cross linking polymer) to a same temperature (e.g., letting reagents sit at room temperature until 25 degrees Celsius); weighing out and separating a specific mass of the cross linking polymer under inert gas (e.g., nitrogen or any other non-oxygen atmosphere gas); determining a ratio of a concentration and a weight of the cross linking polymer to the enzyme; dissolving the weighed amount of enzyme in a desired buffer (e.g., K—HPO4, TRIS, etc.) using, for instance, a magnetic stir bar; dissolving the weighed amount of cross linking polymer under inert atmosphere using, for instance, the magnetic stir bar; centrifuging the solution of enzyme and cross linking polymer at a temperature between 0 to 4 degrees Celsius at low revolutions for between 20 to 60 minutes; adding an equal volume of buffer including additional enzyme to achieve a desired molar ratio of crosslinking polymer:enzyme, and mix using, for instance, the magnetic stir bar; centrifuging the solution of enzyme and cross linking polymer again at same settings for between 20 to 60 minutes; adding increased volume of glycine/buffer mix at increased pH (e.g., 7-12) cold to dilute nanoparticles to, for example, achieve a favorable molar ratio of nanoparticle:crosslinking polymer:enzyme (e.g., a nanoparticle:crosslinking polymer:enzyme ratio of 1:1:1 or 2); centrifuging the solution of enzyme and cross linking polymer again at same settings for between 20 to 60 minutes; and analyzing the solution of enzyme and cross linking polymer through UV-vis at wavelengths of 240 to 340 nms. Through UV-vis spectrophotometer, wavelengths of 240-340 nm show UV presence of proteins (i.e., amino acids) such as tryptophan which demonstrates that the enzyme and the polymer have crosslinked successfully.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

1. A nanoparticle composition, comprising:
a nanoparticle;
at least one polymer; and
at least one protein;
wherein:
manufacturing the nanoparticle composition comprises:
combining a nanoparticle solution comprising the nanoparticle and a solvent with the at least one polymer in a first container to create a first solution; and
combining the first solution with the at least one protein in a second container;
the nanoparticle is selected from a group comprising boron nitride, silica, graphene, cellulose, carbon, latex, silver, and gold;
the at least one polymer is selected from a group comprising polyethylene glycol, polyvinyl alcohol, silanes, paraxylene, and phenol formaldehyde; and
the at least one protein is selected from a group comprising proteinases, kinases, proteases, laccases, and peroxidases.

2. The nanoparticle composition of claim 1, wherein the at least one protein comprises at least one enzyme.

3. The nanoparticle composition of claim 1, wherein the nanoparticle composition comprises a conjugated nanoparticle composition, whereby the at least one protein is conjugated to the nanoparticle to stabilize the at least one protein.

4. The nanoparticle composition of claim 3, wherein manufacturing the nanoparticle composition further comprises:
placing the first container in a sonication bath to mix the nanoparticle solution with the at least one polymer to create the first solution; and
placing the second container in an incubator for a first incubation, wherein the nanoparticle composition is manufactured upon completion of the first incubation.

5. The nanoparticle composition of claim 4, wherein the nanoparticle solution comprises a boron nitride solution.

6. The nanoparticle composition of claim 5, wherein the at least one enzyme comprises at least laccase.

7. The nanoparticle composition of claim 4, wherein:
a component is coated with the nanoparticle composition; and
coating the component with the nanoparticle composition comprises:
placing the component in a third container and submerging the component in the nanoparticle composition; and
placing the third container in an incubator for a second incubation, wherein the component is coated with the nanoparticle composition upon completion of the second incubation.

8. The nanoparticle composition of claim 7, wherein:
an incubation period of the second incubation is between 5 to 48 hours;
an incubation temperature of the second incubation is between 20 to 40 degrees Celsius; and
a pH of the second incubation is between 4 to 7.

9. The nanoparticle composition of claim 1, wherein a textile is comprised of at least the nanoparticle composition.

10. The nanoparticle composition of claim 9, wherein the textile is electrospun from a colloidal suspension comprising the nanoparticle composition.

11. The nanoparticle composition of claim 1, wherein:
the group from which the at least one protein is selected further comprises amylase, cellulase, hemi-cellulase, DNAse, RNAse, pectinase, proteinase, and lipase; and
the group from which the at least one polymer is selected further comprises enzymes, polysaccharides, and DNA/RNA.

12. The nanoparticle composition of claim 1, wherein the nanoparticle composition comprises antimicrobial characteristics for inhibiting growth of micro-organisms.

13. The nanoparticle composition of claim 1, wherein the nanoparticle composition further comprises at least one of:
deflection or attenuating properties for particular types of radiation; and
pores with particular pore sizes for filtering particular chemicals.

14. A process for manufacturing a nanoparticle composition, comprising:
combining a nanoparticle solution comprising a nanoparticle and a solvent with at least one polymer in a first container to create a first solution; and
combining the first solution with at least one protein in a second container;
wherein:
the nanoparticle composition comprises:
the nanoparticle;
the at least one polymer; and
the at least one protein;
the nanoparticle is selected from a group comprising boron nitride, silica, graphene, cellulose, carbon, latex, silver, and gold;
the at least one polymer is selected from a group comprising polyethylene glycol, polyvinyl alcohol, silanes, paraxylene, and phenol formaldehyde; and
the at least one protein is selected from a group comprising proteinases, kinases, proteases, laccases, and peroxidases.

15. The process of claim 14, wherein the nanoparticle composition comprises a conjugated nanoparticle composition, whereby the at least one protein is conjugated to the nanoparticle to stabilize the at least one protein.

16. The process of claim 15, wherein manufacturing the nanoparticle composition further comprises:
placing the first container in a sonication bath to mix the nanoparticle solution with the at least one polymer to create the first solution; and
placing the second container in an incubator for a first incubation, wherein the nanoparticle composition is manufactured upon completion of the first incubation.

17. The process of claim 16, wherein:
the nanoparticle solution comprises a boron nitride solution; and
the at least one enzyme comprises at least laccase.

18. The process of claim 16, wherein:
a component is coated with the nanoparticle composition; and
coating the component with the nanoparticle composition comprises:
placing the component in a third container and submerging the component in the nanoparticle composition; and
placing the third container in an incubator for a second incubation, wherein the component is coated with the nanoparticle composition upon completion of the second incubation.

19. The nanoparticle composition of claim 1, wherein a textile is comprised of at least the nanoparticle composition.

20. The nanoparticle composition of claim 1, wherein the nanoparticle composition comprises at least one of:
antimicrobial characteristics for inhibiting growth of micro-organisms;
deflection or attenuating properties for particular types of radiation; and
pores with particular pore sizes for filtering particular chemicals.
